SEQUENCE LISTING (1 sequence)

SEQ ID NO: 1 - protein, 13 aa, Artificial Sequence
  Rabbit
CSTSKTGHGHAKV

(19) United States
(12) Patent Application Publication
(10) Pub. No.: US 2015/0119476 A1
(43) Pub. Date: Apr. 30, 2015

(54) DEVELOPMENT, CHARACTERIZATION, AND USE OF AN ANTI-HYPUSINATED eIF5A ANTIBODY TO DIAGNOSE DIABETES

(71) Applicant: Indiana University Research and Technology Corp., Indianapolis, IN (US)

(72) Inventor: Raghavendra G. Mirmira, Zionsville, IN (US)

(21) Appl. No.: 14/464,643

(22) Filed: Aug. 20, 2014

Related U.S. Application Data
(60) Provisional application No. 61/867,836, filed on Aug. 20, 2013.

(51) Int. Cl. G01N 33/68; C07K 16/18

(52) U.S. Cl. CPC G01N 33/6872; C07K 16/18; C07K 2317/34; C07K 2317/20; G01N 2800/50; G01N 2800/042; G01N 2333/4703
USPC 514/789; 530/387.9; 436/547; 436/501; 435/7.1

(57) ABSTRACT

Provided herein is a comprehensive characterization of a novel polyclonal antibody (IU-88) that specifically recognizes the hypusinated eIF5A. The antibody IU-88 is useful for the investigation of eIF5A biology, for the development of assays recognizing hypusinated eIF5A, and for methods of treating conditions and diseases that involve the activity of hypusinated eIF5A. The antibody was used to determine that the levels of hypusinated eIF5A were elevated in the pancreatic tissues of patients diagnosed with Type 1 or Type 2 Diabetes.

### CROSS-REFERENCE TO RELATED APPLICATIONS

This application claims priority to U.S. Provisional Patent Application No. 61/867,836 filed on Aug. 20, 2013, the entire disclosure of which is incorporated by reference herein.

### FIELD OF THE INVENTION

Aspects of this invention include an antibody that recognizes hypusinated eIF5A and can be used to study and treat diseases and conditions that involve this eukaryotic translation initiation factor.

### BACKGROUND AND SUMMARY

Eukaryotic translation initiation factor 5A-1 and 5A-2 (eIF5A-1 and eIF5A-2-collectively referred to here as “eIF5A”) are highly conserved proteins whose varied cellular functions include the binding of and nucleocytoplasmic shuttling of specific mRNAs, cellular proliferation, and posttranslational stress responses. Curiously, eIF5A is the only protein thought to include the amino acid hypusine. Hypusinated eIF5A is formed in a posttranslational reaction involving the enzymes deoxyhypusine synthase (DHS) and deoxyhypusine hydroxylase (DHH), the substrate spermidine, and the lysine residue in eIF5A. In the complete absence of deoxyhypusine synthase, mouse embryos die at a very early stage of development. Inhibition of hypusine formation has been suggested to confer cellular survival in certain stress states, such as infections, carcinogenesis, and obesity.

Hypusinated eukaryotic translation initiation factors 5A-1 and 5A-2 have been shown to be responsible for the translation elongation of a subset of cytokine-induced transcripts in 3 cells in the mouse, and it has been shown that eIF5A^{Hyp} also appears to be required for the activation of effector T helper cells. Accordingly, the ability to identify the hypusinated form of eIF5A could prove to be very use in the study of this protein and its role in the cell and pathology in eukaryotic organisms. Some aspects of the invention provide a reagent for the identification of the hypusinated form of eIF5A.

The mechanisms underlying the pathogeneses of Type 1 Diabetes (“T1D”) and Type 2 Diabetes (“T2D”) are thought to involve the activation of systemic and local inflammatory pathways, leading to eventual de-differentiation or death of islet β cells. Accordingly, the identification of biomarkers that can assist in the identification of patients with pre-clinical forms of disease could be very useful for the purposes of early advantageous therapeutic interventions.

Deoxyhypusine synthase (“DHS”) is thought to be the rate-limiting enzyme in the hypusination of eIF5A. The hypusinated form (eIF5A^{Hyp}) is thought to function in mRNA translation elongation and active, for example, in the “stress response.” Cellular processes that have been hypothesized to involve this pathway include inflammation, replication, and cellular differentiation.

Some embodiments include the generation and use of the antibody IU-88, an antibody that recognizes hypusinated eIF5A, or a pharmaceutically acceptable salt thereof. In some embodiments the antibody is produced by the immune system of a rabbit. In some embodiments the antibody is raised against human hypusinated eIF5A. In some embodiments the antibody recognizes a protein that includes the peptide C-Ahx STSKTG[hypusine]HGHAKV-amide (SEQ ID. No. 1).

In some embodiments the inventive antibody is formed by exposing a rabbit to a chimeric protein that includes the peptide C-Ahx-STSKTG[hypusine]HGHAKV-amide (SEQ ID. No. 1). In some embodiments the inventive antibody is a chimeric protein that can be expressed in E. coli.

Still other embodiments include methods for creating an antibody, comprising the steps of: exposing the immune system of a rabbit to a purified protein that includes the polypeptide C-Ahx-STSKTG[hypusine]HGHAKV-amide (SEQ ID. No. 1); and harvesting a polyclonal antibody from the rabbit, wherein said polyclonal antibody recognizes hypusinated eIF5A. In some embodiments the methods for creating an inventive antibody further include the steps of: creating a chimeric protein, wherein the chimeric protein includes the polypetide C-Ahx-STSKTG[hypusine]HGHAKV-amide (SEQ ID. No. 1) and wherein at least one portion of the chimeric protein that includes C-Ahx-STSKTG[hypusine]HGHAKV-amide (SEQ ID. No. 1) is the purified protein exposed to the immune system of the rabbit.

Other embodiments of the invention include methods for detecting hypusinated eIF5A, comprising the steps of: contacting a portion of hypusinated eIF5A, with a polyclonal antibody, wherein the polyclonal antibody recognizes a protein that includes at least one portion of the polypeptide C-Ahx-STSKTG[hypusine]HGHAKV-amide (SEQ ID. No. 1). In some embodiments the polyclonal antibody is from a rabbit. In some embodiment the methods further includes the step of contacting the polyclonal antibody with a second antibody wherein said second antibody recognizes said polyclonal antibody. In some embodiments the second antibody includes at least one label. In some embodiments at least one of the antibodies used to practice the method is labeled with a group selected from the group consisting of: a radioactive atom, a fluorescent moiety, or a chemiluminescent moiety. In some embodiments the protein that includes at least a portion of the polypeptide C-Ahx-STSKTG[hypusine]HGHAKV-amide (SEQ ID. No. 1) is part of a sample from a mammal.

Additionally disclosed is a method for identifying hypusinated eukaryotic translation initiation factor 5A (“eI5A^{Hyp}”) comprising the steps of contacting cells with a polyclonal antibody that recognizes hypusinated eIF5A, or a pharmaceutically acceptable salt thereof and evaluating the contacted cells for expression of eIF5A^{Hyp}. In some embodiments, the contacting step occurs in vitro. In other embodiments, the contacting step occurs in vivo.

In some other embodiments, the cells are human cells. The human cells can be selected from the group of cell types consisting of: spleen CD4+ T cells, spleen Pax5+-expressing B cells, spleen CD8+ T cells, pancreas cells expressing ChgA+, and pancreatic cells expressing pancreatic polypeptide. In some embodiments, the method further comprises the step of comparing the evaluated contacted cells to a control group of cells.

Some embodiment including methods for identifying human or animal patients that have or at risk for developing conditions related to inflammation. Some embodiments include methods for identifying human or animal patients that have or at risk for developing Type 1 or Type 2 Diabetes. Some of these methods include the step of contacting tissue from a patient with an the antibody IU-88 or antibodies with similar reactivity and analyzing the tissue to determine if the cells in the tissue are expressing levels of eIF5A^{Hyp} that are consistent with these diseases or conditions.

Some embodiments include an antibody, which recognizes and binds with selective affinity to hypusinated eIF5A, or a pharmaceutically acceptable salt thereof. In some embodiments the antibody is a monoclonal antibody in some embodiments the antibody is a polyclonal antibody. In some embodiments the antibody the antibody recognizes and binds to a protein that includes the synthetic peptide C-Ahx-STSKTG[hypusine]HGHAKV-amide (SEQ ID. No. 1).

Some embodiments include a method of creating an antibody, comprising the steps of exposing the immune system of an animal such as a rabbit to a polypeptide (preferably a purified polypeptide) that includes at least a portion of the polypeptide sequence C-Ahx-STSKTG[hypusine]HGHAKV-amide (SEQ ID. No. 1); selecting for antibody body that binds selectively to hypusinated eIF5A and harvesting the antibody or at least one cell producing the antibody from the animal. In some embodiments the method of forming the antibody includes creating the polypeptide C-Ahx-STSKTG[hypusine]HGHAKV-amide (SEQ ID. No. 1), in which at least one portion of the protein that includes C-Ahx-STSKTG[hypusine]HGHAKV-amide (SEQ ID. No. 1) and exposing the immune system of the animal to this polypeptide.

Some embodiments further include the step of purifying the polypeptide before injecting the animal with the polypeptide.

Some embodiments include methods for detecting hypusinated eIF5A in vivo and or in vitro present in a purified sample, lysate, whole cell, and or tissue sample. These methods may include the steps of contacting a portion of hypusinated eIF5A, with a monoclonal or polyclonal antibody which reacts with hypusinated eIF5A, wherein the polyclonal antibody recognizes a protein that includes at least one portion of the polypeptide C-Ahx-STSKTG[hypusine]HGHAKV-amide (SEQ ID. No. 1). In some embodiments the methods further includes the step of contacting the bound anti-hypusinated eIF5A antibody with a second antibody wherein said second antibody recognizes the anti-hypusinated eIF5A antibody. In some embodiment the second antibody is includes or is linked to an entity that produces a detectable signal. Such entities include radioactive isotopes, fluorescent groups, and or chemiluminescent groups. In some embodiments the anti-hypusinated eIF5A antibody is contacted with cells or tissues that include cells selected from the group of cell types consisting of: spleen CD4+ T cells, spleen Pax5+-expressing B cells, spleen CD8+ T cells, pancreas cells expressing ChgA+, and pancreatic cells expressing pancreatic polypeptide.

Some embodiments include methods of detecting diseases such as T1D or T2D that include the steps of contacting a sample of pancreatic or other tissue or bodily fluid collected from a patient either diagnosed with T1D or T2D or at risk for developing these conditions with an anti-hypusinated eIF5A antibody and determining the amount of hypusinated eIF5A in the sample relative to a matched sample of tissue taken form control group and or a matched group of patients that have been diagnosed with T1D or T2D. Some embodiments include diagnosing and treating patient thought to have or at risk for developing or identified with at least one disease or condition that includes elevated levels of inflammation relative to disease or condition free matched individuals. In some embodiment the diagnostic method includes identifying patient with a disease or condition based on this analysis. Some embodiments include addition analysis of hypusinated eIF5A over time with and without a therapeutic intervention.

### BRIEF DESCRIPTION OF THE DRAWINGS

FIG. 1A. Immunoblot characterization of polyclonal antibody IU-88. Recombinant human eIF5A was treated in vitro with spermidine, DHS, DHH, and GC7 (300 nM) as indicated, then subjected to polyacrylamide gel electrophoresis and immunoblots analysis using antibody IU-88 or a pan-anti-eIF5A antibody (a BD monoclonal antibody).

FIG. 1B. Recombinant human eIF5A was treated in vitro with spermidine, DHS, and different concentrations of GC7 as indicated, then subjected to polyacrylamide gel electrophoresis and immunoblot analysis using antibody IU-88 or a pan-anti-eIF5A antibody.

FIG. 1C. INS-1 β cells were transfected with a plasmid encoding either GFP-eIF5A(K50A) (lane 1) or GFP-eIF5A (lane 2), then cell extracts were subjected to polyacrylamide gel electrophoresis and immunoblots analysis using antibody IU-88.

FIG. 1D. 293T cells were transfected with a plasmid encoding GFP-eIF5A, with or without another plasmid encoding DHS (as indicated), and treated with different concentrations of GC7 as indicated. Cell extracts were then subjected to polyacrylamide gel electrophoresis and subsequent immunoblots analysis using antibody IU-88, a pan-anti-eIF5A antibody, and an anti-actin antibody.

FIG. 1E. INS-1 cells were transfected with a plasmid encoding GFP-eIF5A, with or without another plasmid encoding DHS (as indicated), and treated with different concentrations of GC7 as indicated. Cell extracts were then subjected to polyacrylamide gel electrophoresis and subsequent immunoblots analysis using antibody IU-88, a pan-anti-eIF5A antibody, and an anti-actin antibody.

FIG. 1F. A diagram illustrating the hypothesis that formation of eIF5A^{Hyp} is part of the cellular stress response.

FIG. 1G. As shown, the process of hypusination requires the polyamine spermidine as substrate, the enzymes DHS and DHH as well as the Lys50 residue of eIF5A.

FIG. 1H. The immunoblot demonstrates the specificity of the anti-eIF5A^{Hyp} antibody (IU-88) to recognize hypusine; however, eIF5A is not hypusinated in cells over-expressing the eIF5A-K50A mutant.

FIG. 2A. Immunocytochemistry of 293T cells using antibody IU-88. 293T cells were transfected with a plasmid encoding GFP-eIF5A, then immunostained using IU-88 and counterstained with DAPI to visualize nuclei. 293T cells were transfected with plasmids encoding GFP-eIF5A and DHS, then immunostained using IU-88 and counterstained with DAPI to visualize nuclei. In both panels A and B, GFP is visualized in the lighter shading. Magnification ×100.

FIGS. 3A-R. Expression of eIF5A^{Hyp} is shown in the spleen of specimens with T1D and T2D.

FIGS. 4A-N. Expression of eIF5A^{Hyp} in the pancreas of a T2D sample is shown. In controls (matched for age, gender, and BMI) and T2D pancreas, expression of eIF5A^{Hyp} was observed in chromograninA (“ChgA”)-expressing cells.

FIGS. 5A-I. Expression of eIF5A^{Hyp} in the pancreas of a T1D sample is shown. Similar to the expression pattern identified in T2D and controls, eIF5AHyp is observed in ChgA-expressing endocrine cells in the T1D (both AAb+ and AAb−) pancreas and controls (matched for age, gender, ethnicity and BMI).

### SEQUENCE LISTING

C-Ahx-STSKTG[hypusine]HGHAKV-amide, SEQ ID NO. 1.

### DETAILED DESCRIPTION

The methods now will be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements.

Likewise, many modifications and other embodiments of the methods described herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation. Unless defined otherwise, all technical and scientific terms used herein have the same meaning as commonly understood by one of skill in the art to which the invention pertains.

The identification of hypusinated eIF5A has remained a challenge, requiring tedious methods such as isoelectric focusing or two-dimensional gel electrophoresis of cellular extracts. Although prior studies reported the development of antibodies against hypusinated eIF5A, their characterizations were limited and utilities of these reagents were not described in subsequent reports. Presented herein is the characterization of a novel anti-hypusine antibody reagent, IU-88. As reported herein, the antibody IU-88 selectively recognizes either the deoxyhypusine or hypusine forms of eIF5A in vitro. Also reported herein is that IU-88 specifically recognizes the hypusinated form of eIF5A in cellular extracts by immunoblots and in whole cells by immunocytochemistry.

### EXPERIMENTAL

### Material and Methods

Cell culture, transfection and DHS inhibition—Human 293T and rat INS-1(832/13) β cells were cultured as described. Cells were transiently transfected with plasmids encoding EGFP-eIF5A, EGFP-eIF5A(K50A) and EGFP-DHS constructs using Lipofectamine 2000 (Invitrogen) for 16 hours before cell extraction or immunofluorescence analysis. The DHS inhibitor GC7 (Biosearch Technologies) was prepared and used in cell culture as previously described.

Reactions in vitro—For in vitro experiments, eIF5A protein was purified from E. coli as a GST fusion, after which the GST tag was proteolytically removed. DHS protein was purified from E. coli as an N-terminal His6 fusion. Purified human DHH protein was purchased from OriGene. The hypusination reactions in vitro proceeded as previously published.

Antibodies and immunoblotting—The rabbit polyclonal antibody IU-88 against hypusinated human eIF5A was generated in rabbits using the synthetic hypusine-containing peptide C-Ahx-STSKTG[hypusine]HGHAKV-amide (SEQ ID NO. 1) by contract to 21^{st} Century Biochemicals. Monoclonal mouse pan-anti-eIF5A antibody was from BD Biosciences and anti-actin antibody was from MP Biomedicals. Immunoblot analysis was visualized using a LiCor Odyssey fluorescence system following electrophoresis on a 4-20% SDS polyacrylamide gel. Primary antibodies were diluted 1:1500 (IU-88) and 1:10,000 (anti-pan-eIF5A).

Fluorescence immunocytochemistry—293T cells were fixed in 4% paraformaldehyde and immunocytochemistry proceeded as previously described. Antibody dilutions were 1:150 for IU-88 and 1:1000 for anti-pan-eIF5A. 4′,6-diamidino-2-phenylindole (DAPI) staining was used to visualize nuclei. A Zeiss LSM-710 microscope was used to visualize cells at magnification ×100.

Analysis of tissue samples from T2D patients—Samples of spleen and pancreatic tissue were collected from patients diagnosed with T2D and from a set of persons matched for age, gender, and Body Mass Index (BMI).

Analysis of tissue samples from TID patients—Samples Tissue samples from patients with T1D included samples from patients with varied histories of the disease. The samples from T1D patients included samples from both autoantibody positive (AAb+) and autoantibody negative (AAb−) patients. Samples from T1D patients were matched with controls; samples from persons matched for age, gender, ethnicity, and Body Mass Index (BMI).

Probing tissue samples-A standard variation of the Fluorescence Immunocytochemistry assay used to create and study the characteristics of the IU-88 antibody was used to probe the tissue samples.

### Results

### Characterization of the IU-88 Anti-eI5A^{Hyp} Antibody.

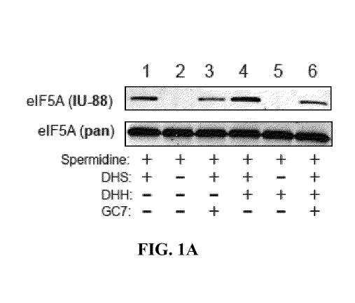

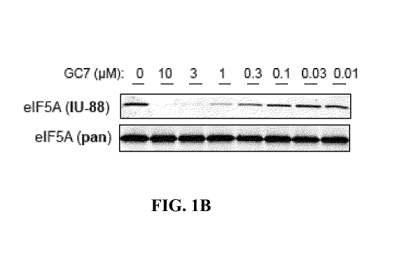

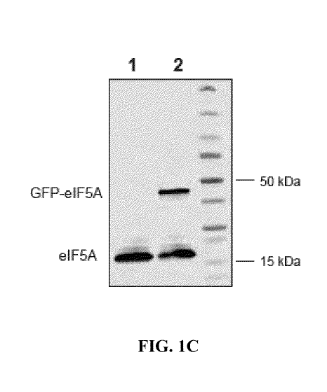

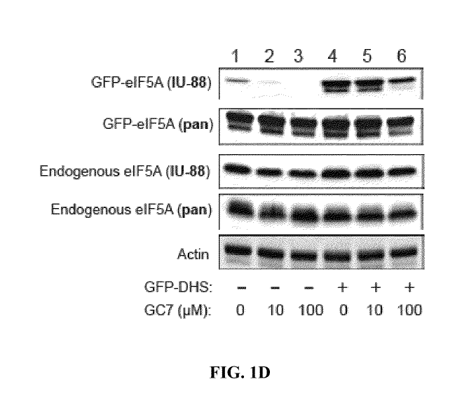

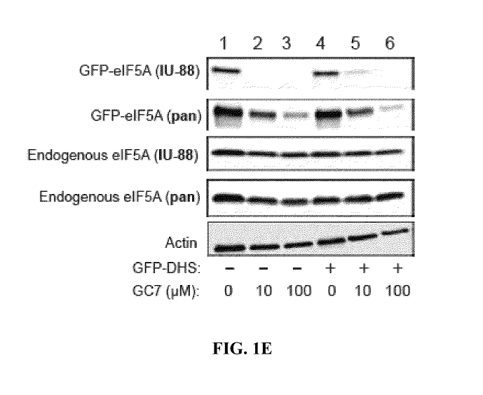

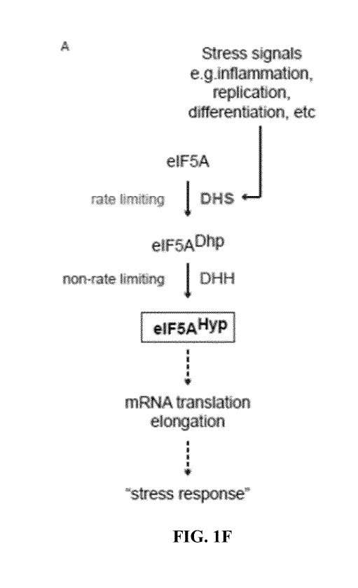

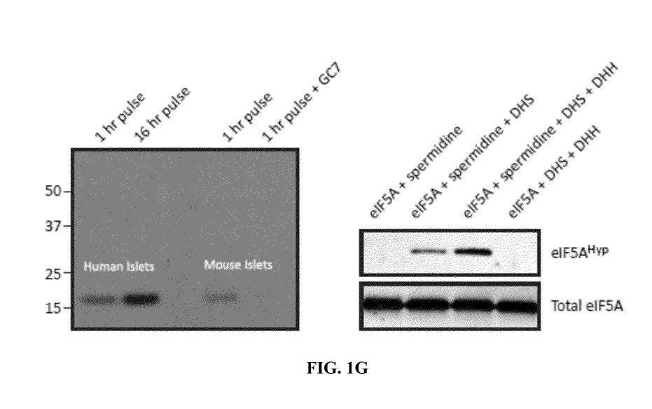

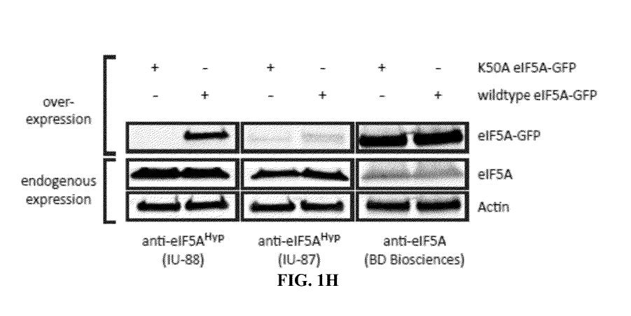

In order to determine if IU-88 specifically recognizes the deoxyhypusine or hypusine forms of eIF5A, immunoblots of reactions in which recombinant human eIF5A was incubated in vitro with DHS, DHH, spermidine, and/or the potent DHS inhibitor GC7 were performed. Referring now to FIG. 1A. FIG. 1A shows that IU-88 is incapable of recognizing eIF5A when it is incubated with spermidine alone (lane 2) or with DHH+spermidine (lane 5) were performed. However, IU-88 recognized eIF5A when co-incubated with DHS+spermidine (lane 1) or DHS+DHH+sperimidine (lane 4), suggesting that both the deoxyhypusine and hypusine forms of eIF5A are recognized. Increasing the DHS concentration and time of incubation led to increasing eIF5A signal intensity in these studies (data not shown). Co-incubation with 300 nM GC7 resulted in a reduction in eIF5A signal intensity (FIG. 1A, lanes 3 and 6), consistent with inhibition of DHS activity by GC7.

Referring now to FIG. 1B. Higher concentrations of GC7 up to 10 μM caused near-complete inhibition of eIF5A signal intensity (FIG. 1B). Notably, the differences in eIF5A intensity in these studies were not because of differences in protein loading, since a pan-anti-eIF5A monoclonal antibody (BD) demonstrated equal loading (FIGS. 1A and B).

The ability of IU-88 to specifically recognize hypusinated eIF5A in whole cellular extract by immunoblotting was tested. Referring now to FIG. 1C. When extracts from rat-derived INS-1 islet β cells are used in immunoblotting, IU-88 recognizes only a single protein species at ˜17 kDa, corresponding to the known molecular weight of eIF5A. Transfection of a plasmid encoding either a human EGFP-eIF5A(K50A) fusion protein (which is not capable of being hypusinated) or a human EGFP-eIF5A fusion protein results in the appearance of a protein species at ˜44 kDa only with the EGFP-eIF5A transfection (FIG. 1C, compared lanes 1 and 2). These data demonstrate specificity of IU-88 in recognizing only eIF5A in total cellular protein, and also suggest that IU-88 only recognizes transfected eIF5A proteins that have the potential to be hypusinated. In order to investigate in greater detail the utility of IU-88 to distinguish hypusination in cellular extracts, additional studies in human-derived 293T cells and INS-1 cells were performed.

Referring now to FIG. 1D. When 293T cells are transfected with GFP-eIF5A, a weak but detectable signal corresponding to GFP-eIF5A is observed using IU-88 (lane 1). This signal decreases further upon co-incubation with increasing concentrations of GC7 (FIG. 1D, lanes 2 and 3), suggesting that IU-88 is recognizing the hypusine-specific form. The endogenous eIF5A signal recognized by IU-88 is also observed to decrease with increasing GC7. Interestingly, when exogenous DHS is introduced by co-transfection of a GFP-DHS fusion protein-encoding vector, there is a dramatic increase in GFP-eIF5A signal (as well as endogenous eIF5A signal) as detected by IU-88 (FIG. 1D, lane 4) with corresponding decrease in the presence of GC7 (lanes 5 and 6), suggesting that DHS protein levels may be limiting in the ability of 293T cells to hypusinate eIF5A-a finding that is also observed in human-derived HeLa cells. INS-1 β cells, by contrast, reveal a significantly different picture.

Referring now to FIG. 1E. Transfection of a plasmid encoding GFP-DHS did not enhance the signal observed with either GFP-eIF5A or endogenous eIF5A, suggesting that DHS is not limiting in the ability of INS-1 cells to hypusinate eIF5A. Interestingly, whereas increasing GC7 concentrations reduces the GFP-eIF5A signal observed with IU-88, it also reduces the signal observed with the pan-anti-eIF5A antibody. This result suggests that INS-1 β cells may be unique in their requirement for hypusination to maintain production of eIF5A itself.

This antibody IU-88 was tested to determine if it could recognize protein in the context of fluorescence immunocytochemistry. 293T cells were transfected with a plasmid encoding GFP-eIF5A, then stained with DAPI (to visualize nuclei) and immunostained using IU-88.

Referring now to FIG. IF. Without being limited to any single hypothesis and solely the diagram presented herewith provide one frame work to understand the etiology and role of eI5A^{Hyp}, Deoxyhupusine synthase (DHS), and the stress response. DHS is the rate-limiting enzyme needed for hypusination of eIF5A, and the hypusinated form (eIF5A^{Hyp}) functions in mRNA translation elongation and the “stress response”. Stress signals hypothesized to invoke this pathway include inflammation, replication, and cellular differentiation.

Referring now to FIG. 1G. The process of hypusination requires the polyamine spermidine as substrate, the enzymes DHS and DHH as well as the Lys50 residue of eIF5A. Using ³H-spermidine and autofluorography it is shown that eIFSA is the only protein that contains the polyamine-derived amino acid, hypusine [N(E)(4-amino-2-hydroxybutyl)lysine]. Using a polyclonal antibody that specifically and reproducibly identifies eIF5A^{Hyp} in an in vitro hypusination assay, it is confirmed that eIF5A^{Hyp} is generated in the presence of DHS and DHH.

Referring now to FIG. 1H, These immunoblots also demonstrate the specificity of the anti-eIF5A^{Hyp} antibody (IU-88) to recognize hypusine; however, eIF5A is not hypusinated in cells over-expressing the eIF5A-K50A mutant.

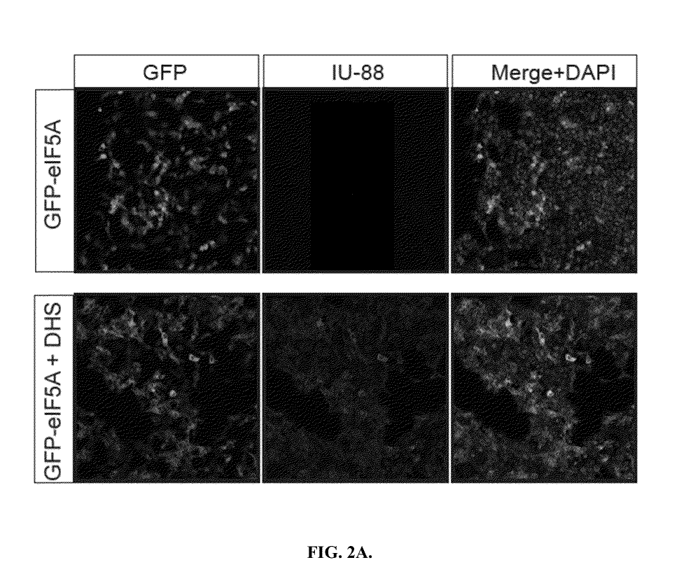

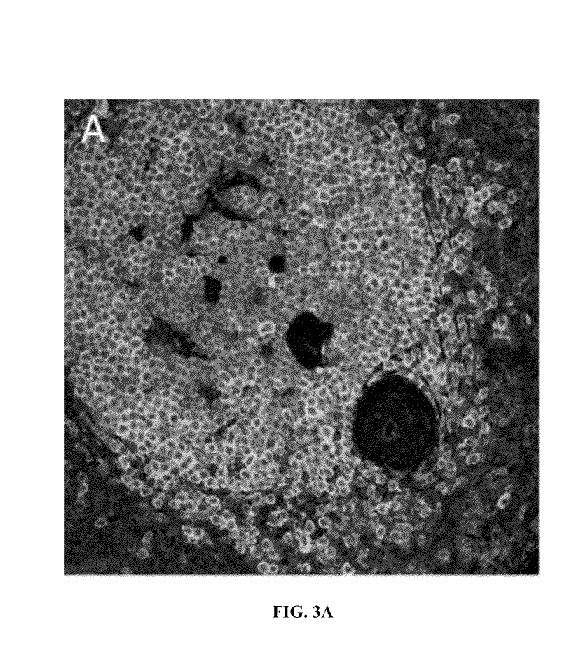

Referring now to FIG. 2A, staining intensity with IU-88 was weak, consistent with the immunoblot in FIG. 1D. However, when cells were co-transfected with GFP-DHS, a striking increase in cytoplasmic staining was observed with IU-88 (FIG. 2A, bottom panel). A notable observation in FIG. 2 is the apparent relocalization of eIF5A from a pan-nuclear/cytoplasmic distribution to a primarily cytoplasmic distribution in the presence of DHS overexpression (c.f. GFP-eIF5A staining in FIG. 2A). This result suggests that hypusinated eIF5A may occupy primarily a cytoplasmic distribution, as proposed in prior studies. However, recent studies have also implicated a role for acetylation in eIF5A compartmentation, suggesting perhaps a more complex interplay between hypusination and other modifications in the function and subcellular localization of the factor.

Taken together, these results verify the specificity and utility of IU-88 in detecting a specifically modified form of eIF5A. IU-88 bind to both the deoxyhypusinated and hypusinated forms of eIF5A. Although the relative significance of the deoxyhypusinated vs. hypusinated forms of eIF5A remains unclear, the low substrate Km of DHH relative to DHS means that the majority of eIF5A in cells is likely present in the fully hypusinated form. The antibody IU-88 represents an especially useful reagent for the assessment of at least the activity of DHS in cells. Also, because most pharmacologic approaches to inhibiting the hypusination reaction have focused on inhibition of the higher Km enzyme DHS, IU-88 would also serve as an important reagent for assessing DHS activity in drug screening studies.

Probing Human Tissue Samples for the Presence of eI5A^{Hyp}.

In one experiment, the novel polyclonal antibody discussed above was used with human tissue samples to determine if the presence of eIF5A^{Hyp} marks a significant population of cells in the human pancreas, and whether or not this population of cells stratifies with characteristics of disease. A collection of pancreas and spleen from persons with T2D and controls matched for age, gender and BMI were analyzed. Additionally, pancreas and spleen from persons with T1D were also evaluated. T1D cases varied in duration of disease and included both autoantibody positive and autoantibody negative samples, and controls matched for age, gender, ethnicity and BMI.

In spleen, eIF5A^{Hyp} expression was observed in CD4+ cells and in Pax5+ B cells, but was largely excluded from CD8+ cells; no difference in staining intensity or distribution was observed between samples from T2D, T1D, and controls. The patterns of expression were substantially the same in all cases analyzed. In the pancreas, a population of eIF5A^{Hyp}+/ChgA+ cells was identified in the islets of T2D and T1D, which may be increased in frequency compared with corresponding controls. The expression of eIF5A^{Hyp} in beta (insulin), alpha (glucagon), or epsilon (ghrelin) cells was not observed. The eIF5A^{Hyp+}/ChgA+ cells also appear to express the hormone pancreatic polypeptide; co-expression with other islet hormones was not observed in the experiment.

Without being limited to any one theory or explanation, the results in one experiment indicate a cell-specific enrichment of eIF5A^{Hyp} in populations of immune cells in the spleen and endocrine cells in the pancreas. The frequency of these cells in the pancreas may be increased in diabetic states.

Pattern of eI5A^{Hyp} Expression in Spleen Tissue from Patients Diagnosed with T1D or T2D Relative to Tissue Samples from Matched Controls.

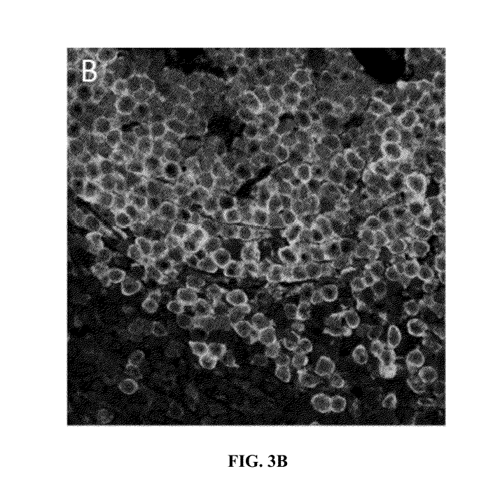

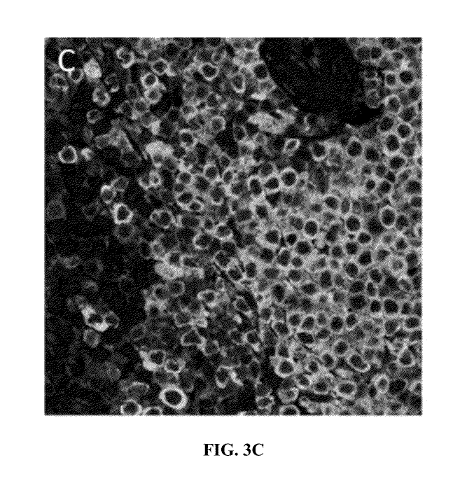

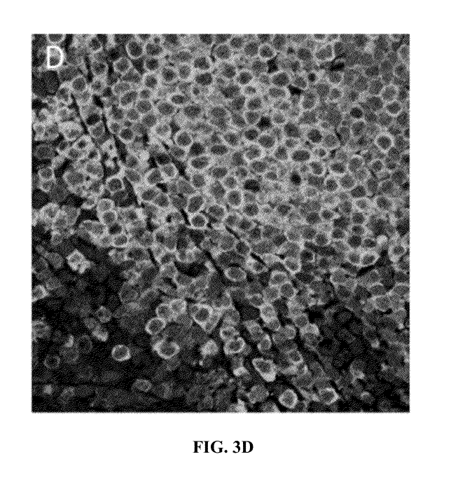

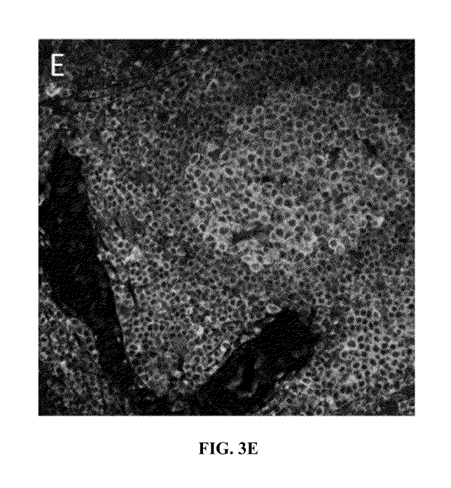

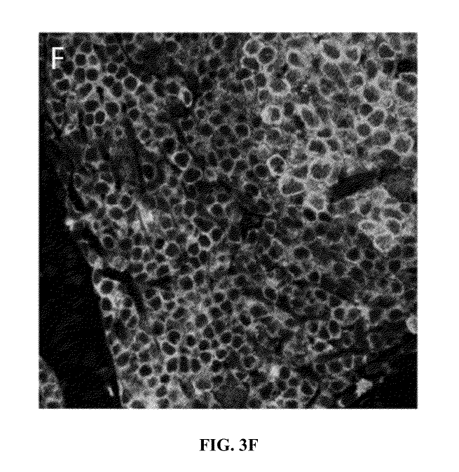

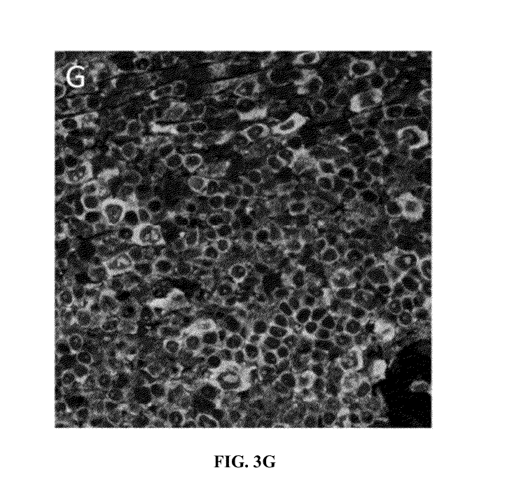

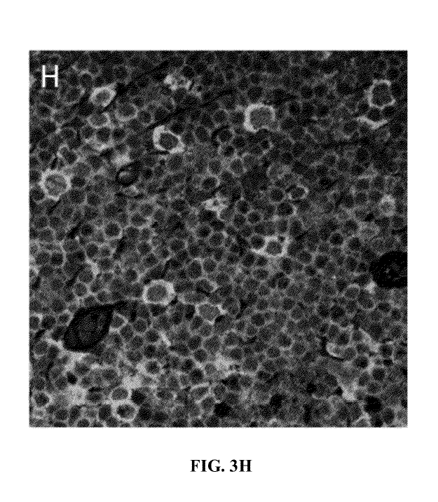

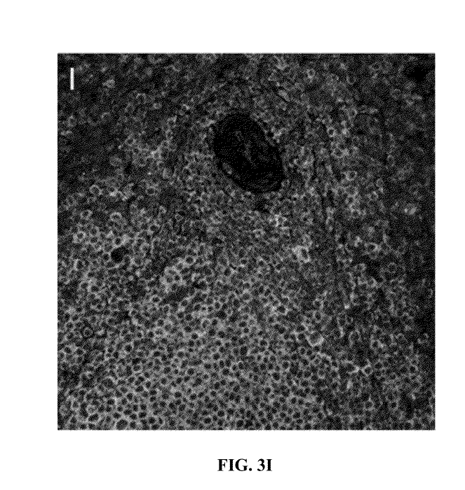

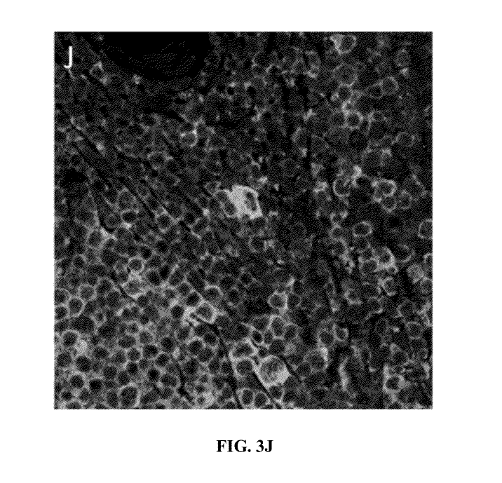

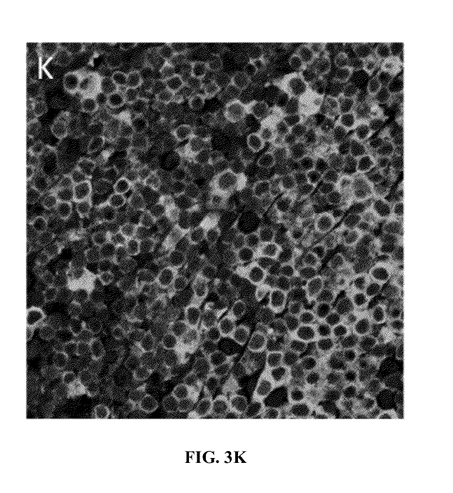

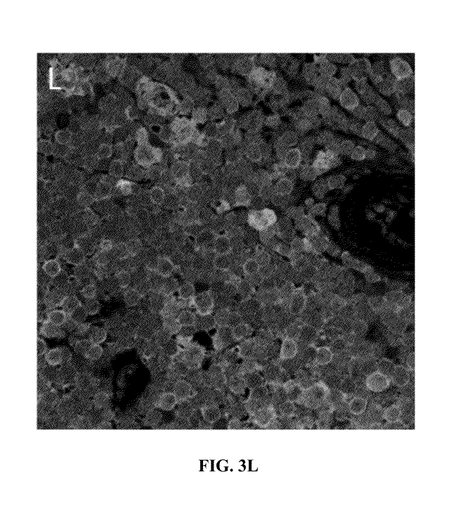

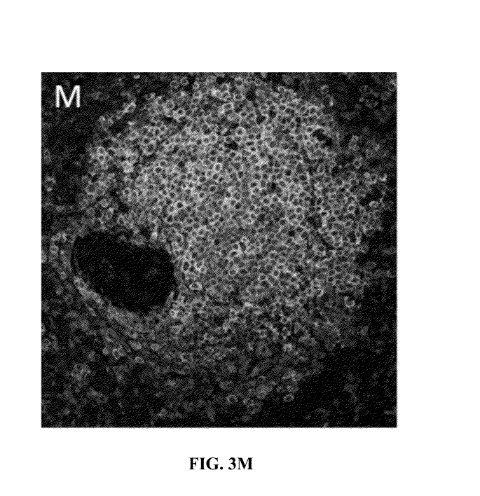

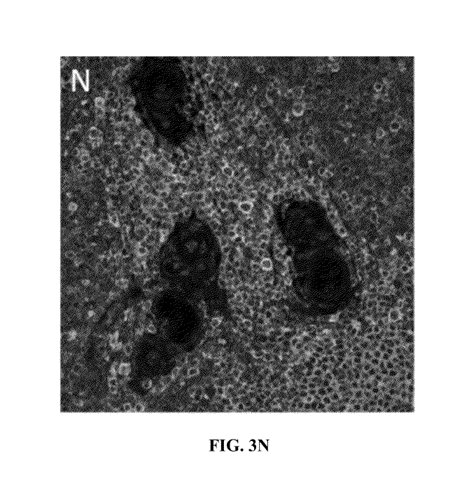

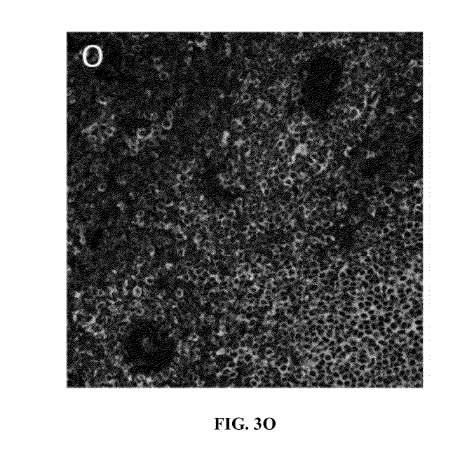

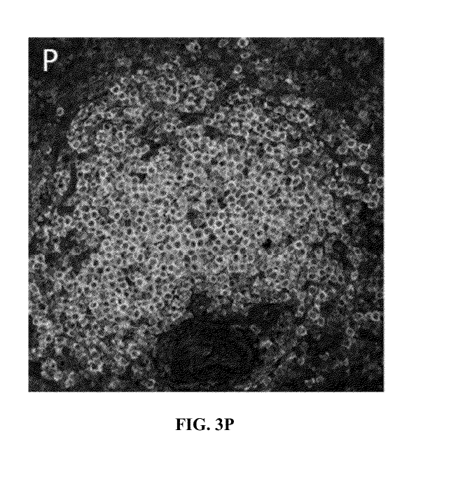

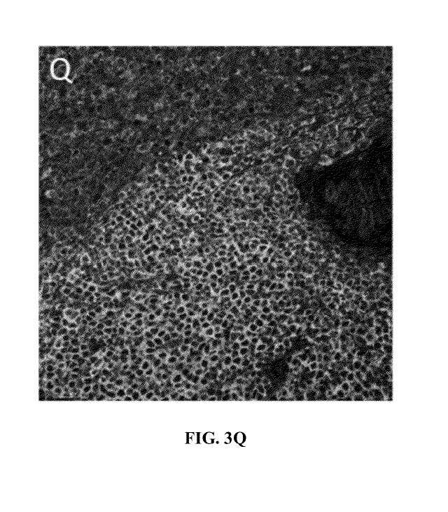

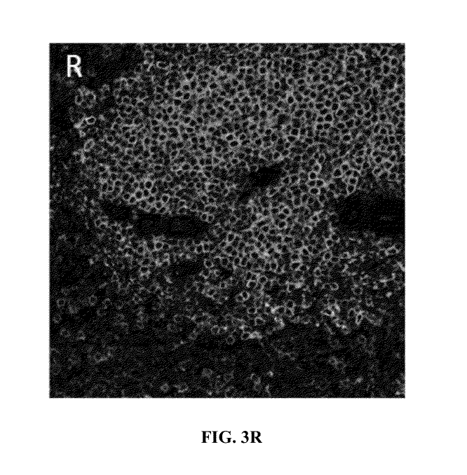

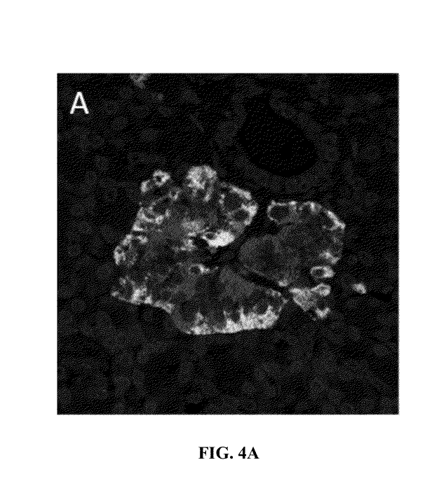

Referring now to FIGS. 3A-R, expression of eIF5A^{Hyp} is shown in the spleen of specimens with T1D and T2D. The lightest shading pictured in FIGS. 3A-R show that eIF5A^{Hyp} is expressed in immune cells in the spleen. Spleen tissue from persons with autoantibody (+) (AAb+) and autoantibody(−) (AAb−) T1D were examined, and corresponding controls were matched for age, gender, ethnicity and BMI. The expression of eIF5A^{Hyp} was evaluated in Pax5+B cells, CD4+ T cells and CD8+ T cells.

FIGS. 3A-D show that most eIF5A^{Hyp}+ cells co-expressed Pax5+. FIG. 3A shows eIF5A^{Hyp}/Pax5/DAPI in a control sample magnified 20×. DAPI is 4′,6-diamidino-2-phenylindole, a DNA-specific probe which forms a fluorescent complex by attaching in the minor grove of A-T rich sequences of DNA. It also forms nonfluorescent intercalative complexes with double-stranded nucleic acids.

FIG. 3B shows eIF5A^{Hyp}/Pax5/DAPI in a control sample. FIG. 3C shows eIF5A^{Hyp}/Pax5/DAPI in a T1D (AAb+) sample. FIG. 3D shows eIF5A^{Hyp}/Pax5/DAPI in a T1D (AAb−) sample.

FIGS. 3E-H show that a select group of eIF5A^{Hyp}+ cells expressed CD4+. FIG. 3E shows eIF5A^{Hyp}/CD4/DAPI in a control sample magnified 20×. FIG. 3F shows eIF5A^{Hyp}/CD4/DAPI in a control sample. FIG. 3G shows eIF5A^{Hyp}/CD4/DAPI in a T1D (AAb+) sample. FIG. 3H shows eIF5A^{Hyp}/CD4/DAPI in a T1D (AAb−) sample.

FIGS. 3I-L show that a select group of eIF5A^{Hyp}+ cells expressed CD8+. FIG. 3I shows eIF5A^{Hyp}/CD8/DAPI in a control sample magnified 20×. FIG. 3J shows eIF5A^{Hyp}/CD8/DAPI in a control sample. FIG. 3K shows eIF5A^{Hyp}/CD8/DAPI in a T1D (AAb+) sample. FIG. 3L shows eIF5A^{Hyp}/CD8/DAPI in a T1D (AAb−) sample.

Referring now to FIGS. 3M-R, spleen samples from T2D and controls matched for age, gender, and BMI were also evaluated for eIF5A^{Hyp}. FIG. 3M shows eIF5A^{Hyp}/Pax5/DAPI in a control sample. FIG. 3N shows eIF5A^{Hyp}/CD4/DAPI in a control sample. FIG. 3O shows eIF5A^{Hyp}/CD8/DAPI in a control sample.

FIG. 3P shows eIF5A^{Hyp}/Pax5/DAPI in a T2D sample. FIG. 3Q shows eIF5A^{Hyp}/CD4/DAPI in a T2D sample. FIG. 3R shows eIF5A^{Hyp}/CD8/DAPI in a T2D sample. The expression pattern of eIF5A^{Hyp} in the spleen of T2D (FIGS. 3P-R) and in the controls (FIGS. 3M-0) was substantially similar to that observed in the T1D spleen samples (FIGS. 3A-L).

Pattern of eI5A^{Hyp} Expression in Pancreatic Tissue from Patients Diagnosed with T1D or T2D Versus Samples Ofpancreatic Tissue from Matched Controls

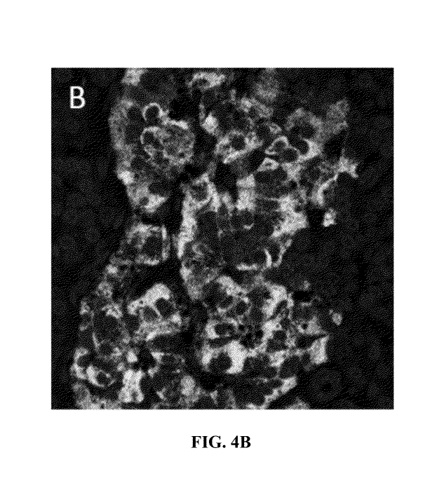

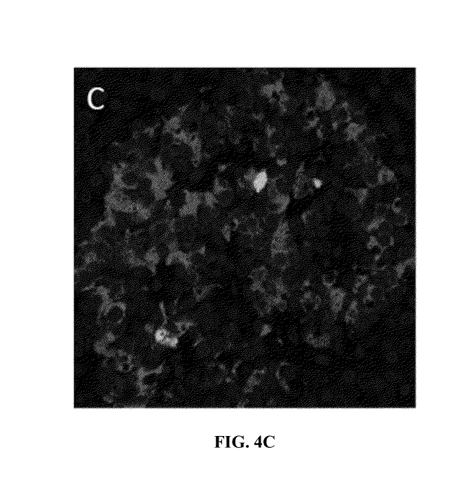

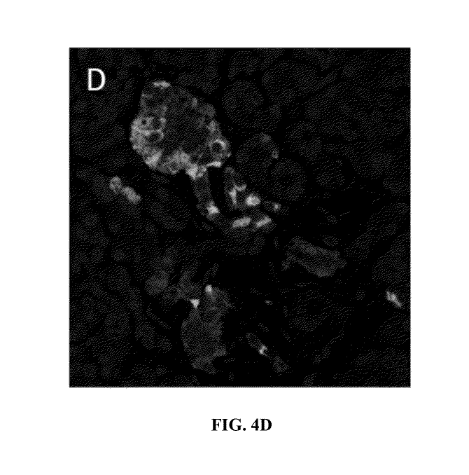

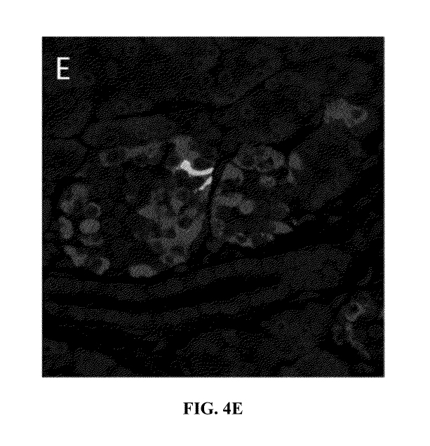

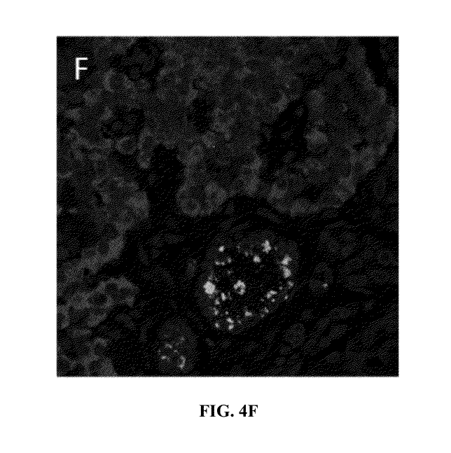

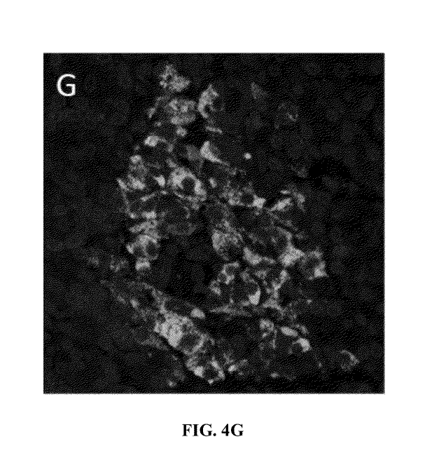

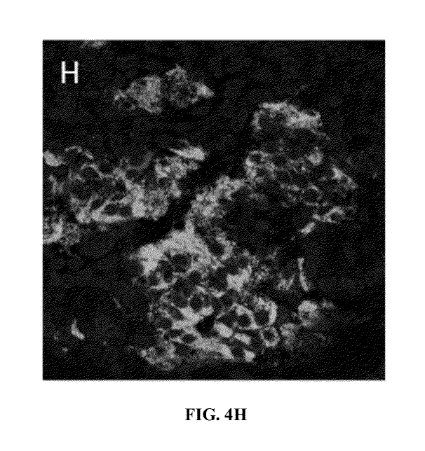

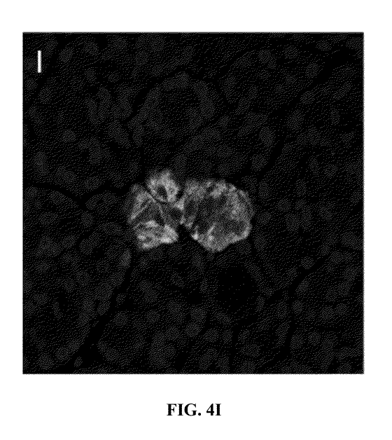

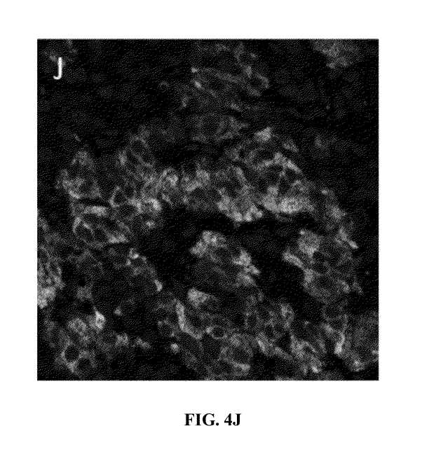

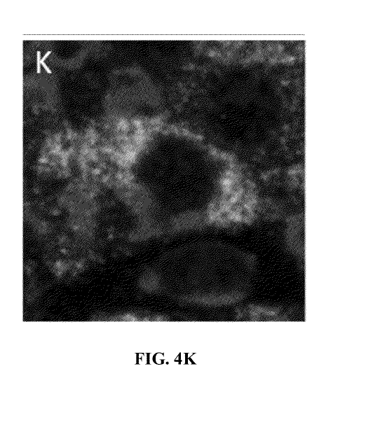

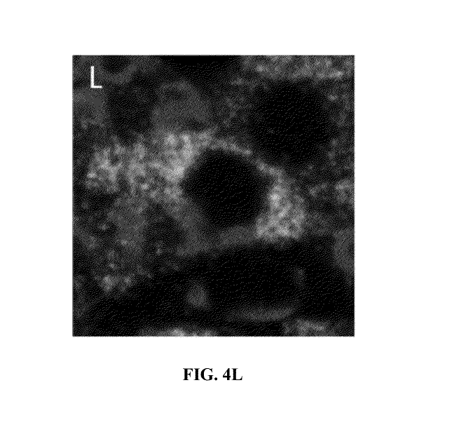

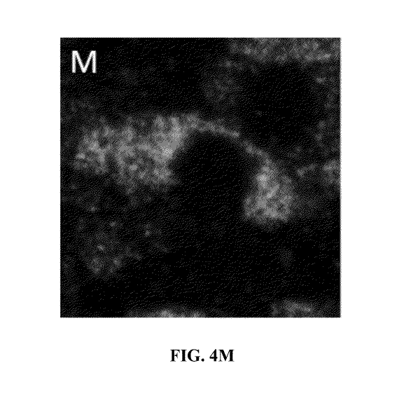

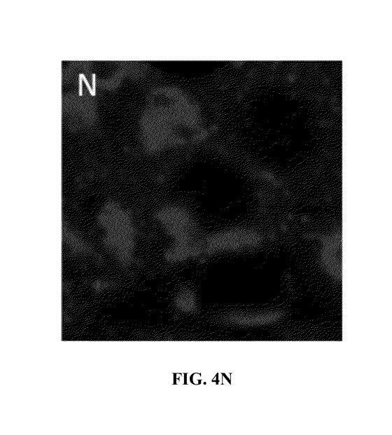

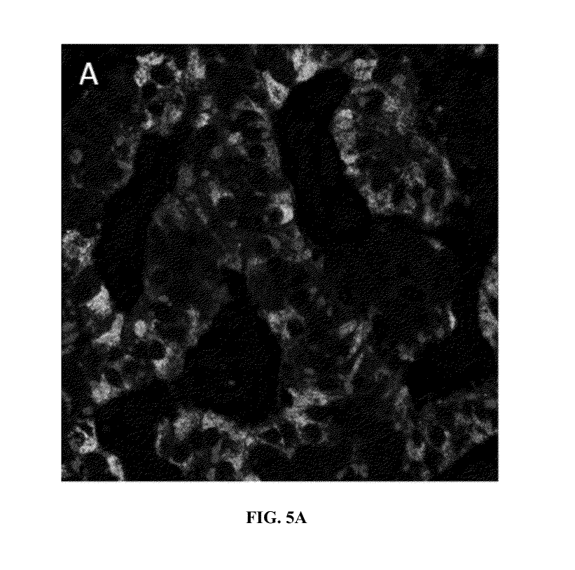

Referring now to FIGS. 4A-N, expression of eIF5A^{Hyp} in the pancreas of a T2D sample is shown. In controls (matched for age, gender, and BMI) and T2D pancreas, expression of eIF5A^{Hyp} was observed in chromograninA (“ChgA”)-expressing cells. FIG. 4A shows eIF5A^{Hyp}/ChgA/DAPI in a control sample, and FIG. 4B shows eIF5A^{Hyp}/ChgA/DAPI in a T2D sample. The lightest shading in the figures shows the location of eIF5A^{Hyp}.

Pancreatic tissue was further analyzed for co-expression of eIF5A^{Hyp} with specific islet hormones. FIG. 4C shows eIF5A^{Hyp}/insulin/DAPI in a control sample, and FIG. 4D shows eIF5A^{Hyp}/insulin/DAPI in a T2D sample. FIG. 4E shows eIF5A^{Hyp}/glucagon/DAPI in a control sample, and FIG. 4F shows eIF5A^{Hyp}/glucagon/DAPI in a T2D sample. FIG. 4G shows eIF5A^{Hyp}/ghrelin/DAPI in a control sample, and FIG. 4H shows eIF5A^{Hyp}/ghrelin/DAPI in a T2D sample. FIG. 4I shows eIF5A^{Hyp}/pancreatic polypeptide/DAPI in a control sample, and FIG. 4J shows eIF5A^{Hyp}/pancreatic polypeptide/DAPI in a T2D sample. Only pancreatic polypeptide cells (FIGS. 4I-J) were identified to express eIF5A^{Hyp}.

FIGS. 4K-N show magnified images of pancreatic polypeptide (“PP”)-expressing PP cells co-expressing eIF5A^{Hyp} in the pancreas of persons with T2D; corresponding controls not shown display a substantially similar staining pattern. The lightest shading shows eIF5A^{Hyp}. FIG. 4K shows enlarged eIF5A^{Hyp}/PP/DAPI, FIG. 4L shows enlarged eIF5A^{Hyp}/PP, FIG. 4M shows enlarged eIF5A^{Hyp} alone, and FIG. 4N shows enlarged PP alone.

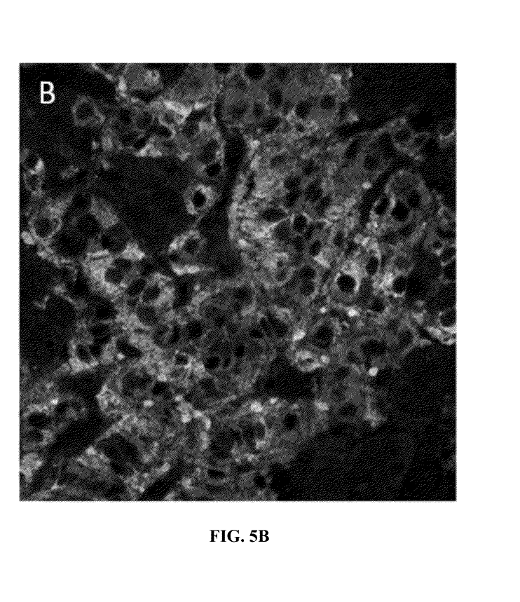

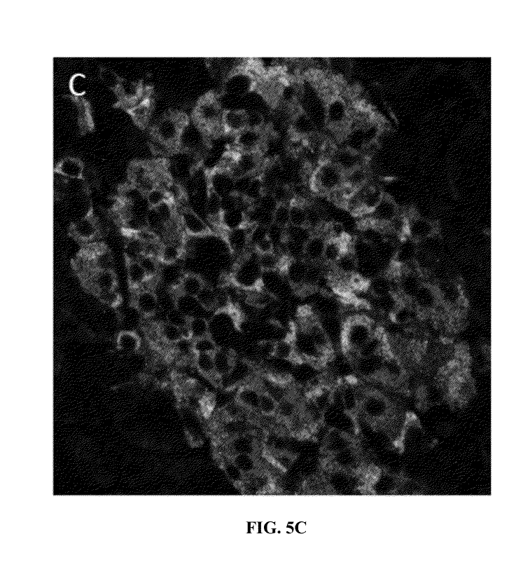

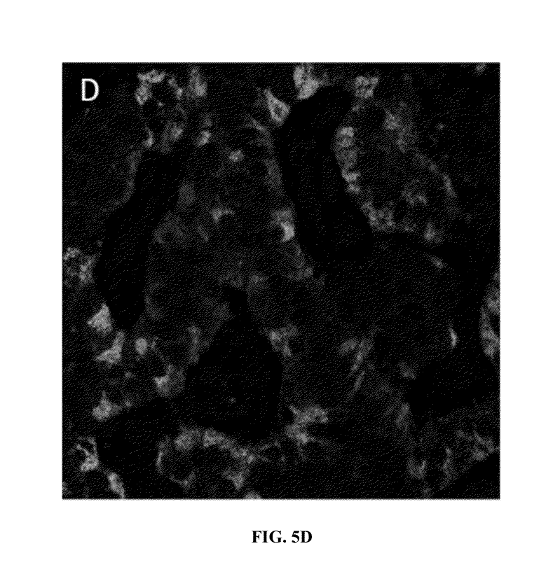

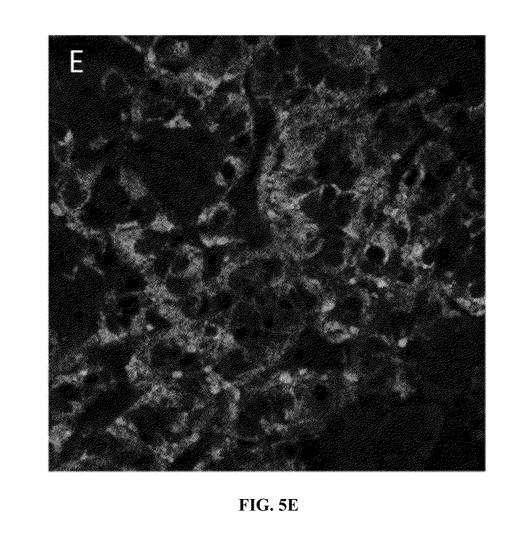

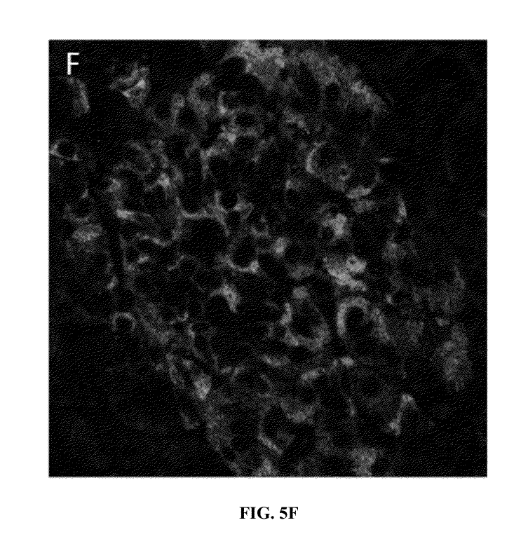

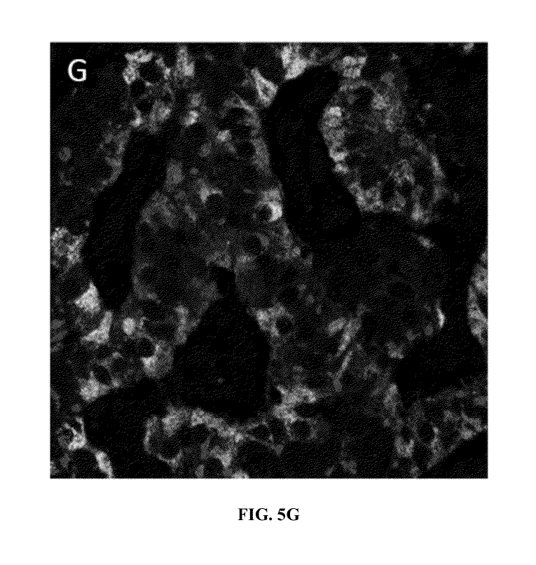

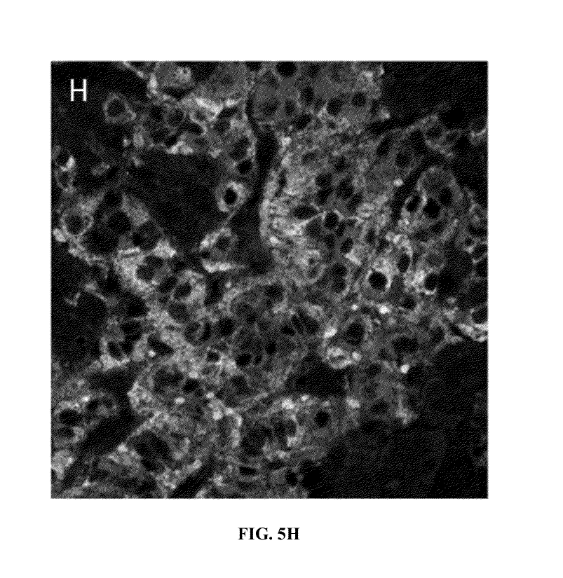

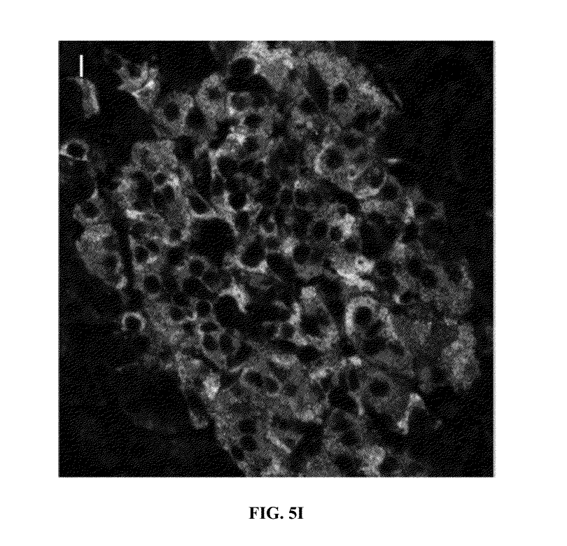

Referring now to FIGS. 5A-I, expression of eIF5A^{Hyp} in the pancreas of a T1D sample is shown. Similar to the expression pattern identified in T2D and controls, eIF5AHyp is observed in ChgA-expressing endocrine cells in the T1D (both AAb+ and AAb−) pancreas and controls (matched for age, gender, ethnicity and BMI). As FIGS. 5A-I show, PP cells are islet cells that express eIF5A^{Hyp}. The number of eIF5A^{Hyp}-expressing cells appears greater in the T1D (AAb+) and T1D (AAb−) tissue compared with controls. While not shown in the Figures, it has been evaluated whether eIF5A^{Hyp} is co-expressed with insulin or glucagon in the T1D and control samples, and the expression of eIF5A^{Hyp} has not been observed in beta (insulin) or alpha (glucagon) cells.

FIG. 5A shows eIF5A^{Hyp}/PP in a control, FIG. 5B shows eIF5A^{Hyp}/PP in T1D (AAb+) and FIG. 5C shows eIF5A^{Hyp}/PP in T1D (AAb−). The lightest shading is used to show eIF5A^{Hyp}. FIG. 5D shows eIF5A^{Hyp} in a control, FIG. 5E shows eIF5A^{Hyp} in T1D (AAb+) and FIG. 5F shows eIF5A^{Hyp} in T1D (AAb−). FIG. 5G shows eIF5A^{Hyp}/PP/ChgA in a control, FIG. 5H shows eIF5A^{Hyp}/PP/ChgA in T1D (AAb+) and FIG. 5I shows eIF5A^{Hyp}/PP/ChgA in T1D (AAb−).

In spleen, eIF5A^{Hyp} expression was observed in CD4+ cells and in Pax5+ cells, but was largely excluded from CD8+ cells; no differences in staining intensity or distribution were observed between samples from T2D, T1D, and controls. In the pancreas, we identified a population of eIF5A^{Hyp}+/ChgA+ cells in the islets of T2D and T1D, which may be increased in frequency compared with corresponding controls. These eIF5A^{Hyp}+/ChgA+ cells appear to also express the hormone pancreatic polypeptide; co-expression with other islet hormones was not observed. These results are consisting with an enrichment of eIF5A^{Hyp} in populations of immune cells in the spleen and endocrine cells in the pancreas. Moreover, the frequency of these cells in the pancreas may be increased in diabetic states.

An enrichment of eIF5A^{Hyp} in Pax5-expressing B cells, as well as a subset of CD4+ and CD8+ T cells was revealed in the spleen of persons with T1D, T2D and corresponding controls. The patterns of expression were identical in each case analyzed.

In the pancreas of T1D, T2D and corresponding controls it was identified that eIF5A^{Hyp} is expressed in endocrine cells, i.e. those expressing ChgA. However, the expression of eIF5A^{Hyp} in beta (insulin), alpha (glucagon), or epsilon (ghrelin) cells was not observed. Rather eIF5A^{Hyp} expression was identified in cells expressing pancreatic polypeptide (PP) in all cases evaluated. The frequency of these cells in the pancreas may be increased in diabetic states.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

We claim:

1. An antibody, comprising:
an anti-hypusinated eIF5A antibody, or a pharmaceutically acceptable salt thereof.

2. The antibody according to claim 1, wherein the antibody is raised against human hypusinated eIF5A.

3. The antibody according to claim 1, wherein said antibody recognizes a protein that includes the synthetic peptide C-Ahx-STSKTG[hypusine]HGHAKV-amide (SEQ ID. No. 1).

4. The antibody according to claim 1, wherein said antibody is produced by the immune system of an animal selected from the group consisting of rabbits, mice, goats, horse, and humans.

5. The antibody according to claim 1, wherein said antibody is a rabbit polyclonal antibody.

6. A method of creating an antibody, comprising the steps of:
exposing the immune system of an animal to a protein that includes the polypeptide C-Ahx-STSKTG[hypusine]HGHAKV-amide (SEQ ID. No. 1); and
harvesting an anti-hypusinated eIF5A antibody from the animal wherein said antibody recognizes hypusinated eIF5A.

7. The method according to claim 6, wherein the antibody is a polyclonal antibody.

8. The method according to claim 6, wherein the animal is a rabbit.

9. The method according to claim 6, further including the steps of:
creating a chimeric protein, wherein the chimeric protein includes the polypetide C-Ahx-STSKTG[hypusine]HGHAKV-amide (SEQ ID. No. 1) and wherein at least one portion of the chimeric protein that includes C-Ahx-STSKTG[hypusine]HGHAKV-amide (SEQ ID. No. 1) is the purified protein exposed to the immune system of the animal.

10. A method for detecting hypusinated eIF5A, comprising the steps of:
contacting at least a portion of a hypusinated form of eIF5A, with an anti-hypusinated eIF5A antibody, wherein the antibody recognizes a protein that includes at least one portion of the polypeptide C-Ahx-STSKTG[hypusine]HGHAKV-amide (SEQ ID. No. 1).

11. The method according to claim 10, wherein the antibody is a polyclonal antibody from a rabbit.

12. The method according to claim 10, wherein the method further includes the step of contacting the anti-hypusinated eIF5A antibody with a second antibody wherein said second antibody recognizes the anti-hypusinated eIF5A antibody.

13. The method according to claim 10, wherein the second antibody includes at least one entity that produces a detectable signal.

14. The method according to claim 10, wherein the at least one entity that produces a detectable signal is selected from the group consisting of: a radioactive isotope, a fluorescent moiety, or a chemiluminescent moiety.

15. The method according to claim 10, wherein the hypusinated eIF5A is present in a sample selected from the group consisting of bodily fluids, tissue, and cells.

16. The method according to claim 15, further including the steps of;
determining a level of hypusinated eIF5A in a sample from a patient;
comparing the level of hypusinated eIF5A in the sample from the patient with a level of hypusinated eIF5A determined in a matched sample from a matched person or group of persons; and
identifying the patient as having a disease or a risk for developing a disease if the level of hypusinated eIF5A in the sample from the patient is elevated relative to the level of hypusinated eIF5A in the matched person of groups of persons.

17. The method according to claim 15, wherein the disease exhibits pathological inflammation.

18. The method according to claim 15, wherein the sample is a portion of pancreatic tissue and wherein the patient is identified as having Diabetes or being at risk for developing the symptoms of Diabetes if the level of hypusinated eIF5A in the sample from the patient is elevated relative to the level of hypusinated eIF5A in the matched control group.

19. The method according to claim 15, wherein the sample is a portion of pancreatic tissue and wherein the patient is identified as having Diabetes or being at risk for developing the symptoms of Diabetes if the level of hypusinated eIF5A in the sample from the patient is equivalent to the level of hypusinated eIF5A in a matched person of group of matched person that have been diagnosed has having Diabetes or of being at risk for developing Diabetes.

20. The method according to claim 15, further including the step of treating the patient for a disease or a condition if the patient has been identified has having the disease or the condition.
